(19) United States
(12) Patent Application Publication
(10) Pub. No.: US 2003/0033010 A1
(43) Pub. Date: Feb. 13, 2003

(54) Method of improved keratoprosthesis

(76) Inventors: Celia R. Hicks, Nedlands, WA (US); Traian V. Chirila, Nedlands, WA (US); Geoffrey J. Crawford, Nedlands, WA (US); Xia Lou, Nedlands, WA (US)

Correspondence Address: PALMER & DODGE, LLP, KATHLEEN M. WILLIAMS, 111 HUNTINGTON AVENUE, BOSTON, MA, 02199, US

(21) Appl. No.: 10/167,875

(22) Filed: Jun. 12, 2002

Related U.S. Application Data
(60) Provisional application No. 60/297,952, filed on Jun. 13, 2001.

(51) Int. Cl. A61F 2/14

(52) U.S. Cl. 623/5.11; 623/5.16; 606/107

(57) ABSTRACT

The invention relates to an improved keratoprosthesis comprising a keratoprosthesis prepared with such properties and radii that when implanted into a patient it assumes the optic radii of curvature for a desirable refractive outcome.

### RELATED APPLICATIONS

This application claims the benefit of U.S. Provisional Application Ser. No. 60/297,952, filed Jun. 13, 2001. The entire teachings of the above application is incorporated herein by reference.

### FIELD OF THE INVENTION

This invention relates generally to an improved keratoprosthesis and to methods for its production.

### BACKGROUND ART

The cornea is not only the major component of the optical system of the eye by providing about 75% of the total dioptric power, but also serves a protective function against the external environment. In order to perform its normal functions (refraction, transmission, protection), the cornea must actively maintain its transparency and integrity throughout life.

Replacement of an injured and/or opaque cornea is most commonly done through transplantation of donor corneal tissue. Such replacements are often successful in less complex pathological conditions, such as keratoconus or corneal dystrophies, although the success rate falls with time. However, certain conditions are associated with much lower success rates and with repeat grafts the chance of a graft remaining clear drops significantly. Additionally, the visual acuity outcome is often disappointing due to refractive errors, even in cases where the donor graft tissue retains its clarity. Further, even in many countries with an organised eye banking system, there is a chronic shortage of donor corneal tissue.

The alternate approach to corneal replacement involves the use of prosthetic corneas, known as keratoprostheses, or, if bearing closer resemblance to donor corneal buttons, sometimes called artificial corneas, the surgery being generally known as prosthokeratoplasty. Traditional keratoprostheses are generally associated with high complication rates, are cosmetically disfiguring and may have restricted visual outcomes. They are generally reserved for severely debilitating bilateral corneal disease and are unsuitable for use outside certain specialist centres. Artificial corneas that have demonstrably lower complication rates and wider indications are now becoming more widely used, making predictable and satisfactory visual outcomes for recipients an important goal.

Patients undergoing corneal replacement of any kind may be aphakic, i.e. have no natural biological crystalline lens in place in the eye, phakic i.e. retain their biological crystalline lens, or pseudophakic, i.e. have had their natural crystalline lens previously replaced by a synthetic intraocular lens, due to the natural lens having become cataractous. The optical requirements of these different patient categories differ significantly.

Australian patent 650156, and U.S. Pat. Nos. 5,300,116 and 5,458,819, assigned to the present applicant, describe a keratoprosthesis and a method for its production. The prosthesis generally disclosed in these patents is a one-piece “core and skirt” device, comprised of intimately attached central and peripheral portions. The central portion or core is a transparent lenticular part and is a hydrogel composed essentially of a biocompatible hydrophilic polymer, while the peripheral portion, a porous annular skirt, is composed essentially of a like polymer but is a porous hydrogel sponge. A suitable material for both parts is, for example, a polymer of 2-hydroxyethylmethacrylate (commonly designated as “PHEMA”). The spongy periphery promotes and maintains cellular invasion from the host corneal tissue, thus providing a tight union between implant and recipient cornea that prevents the post-operative extrusion of the implant. Other unitary and composite keratoprostheses have been suggested in a wide range of references and have generally utilised materials such as poly(methylmethacrylate) (PMMA), polytetrafluoroethylene (PTFE or Teflon®), silicones and polyurethanes.

It will be appreciated that, ideally, the implantation of such a keratoprosthesis in a patient for whom the procedure is considered beneficial would be preceded by the selection or customised moulding of a prosthesis having the specific optical requirements for that individual, necessitating either an extensive range of pre-manufactured prostheses to provide a wide selection, or access to custom moulding services. In practice, such individualisation of supply is difficult to provide and keratoprosthesis recipients, like standard donor graft recipients, tend to receive a ‘standard’ implant and may be left with significant refractive errors for post-operative management.

Prior art keratoprostheses have depended upon a range of standard corrective methods for refractive errors, including contact lenses and spectacles, to rectify these problems but in many cases the uncorrected visual acuity is extremely limited.

A limitation of current keratoprostheses is that it is not practically possible to provide an individualised keratoprosthesis for every patient. It is however, desirable to prepare a keratoprosthesis that will give a ‘reasonable’ visual acuity on implantation and then to provide an individually tailored post-operative correction using any combination of methods of refractive correction known to persons skilled in the art of refractive correction.

The present invention seeks to provide a means for improving upon prior art keratoprostheses by preparation of keratoprostheses that provide a power rating suitable for a standardised phakic/pseudophakic or aphakic eye in situ that is then easily adjusted to individual requirements after implantation. Further, such an artificial cornea, by providing optical outcomes as good as, or better than, those obtained through donor tissue grafting, but without the associated risks and disadvantages of using donor human tissue, would have a wide application beyond the restricted aetiologies generally considered suitable for traditional keratoprostheses.

The preceding discussion of the prior art is intended to facilitate an understanding of the present invention only. It should be appreciated that the discussion is not an acknowledgement or admission that any of the material referred to was part of the common general knowledge as at the priority date of the application.

### SUMMARY OF THE INVENTION

The present invention seeks to provide a means for improving upon prior art keratoprostheses by preparation of keratoprostheses that have a power rating suitable for a standardised phakic/pseudophakic or aphakic eye in situ that is then easily adjusted to individual requirements after implantation.

According to one form of the invention, there is provided an improved keratoprosthesis comprising a keratoprosthesis possessing such properties and radii that when implanted into a patient it assumes the optic radii of curvature for a desirable refractive outcome. Generally, a desirable refractive outcome would be regarded as a refractive power in the order of approximately 42 D in situ in phakic or pseudophakic patients and appropriately higher in aphakic recipients.

Such an artificial cornea, by providing optical outcomes as good as, or better than, those obtained through donor tissue grafting, but without the associated risks and disadvantages of using donor human tissue, will have wide application beyond the restricted aetiologies generally considered suitable for traditional keratoprostheses.

According to a second form of the invention, there is provided a method of implanting a keratoprosthesis into a patient, said method comprising the steps of:

(a) Forming a lamellar corneal pocket;

(b) Providing a posterior lamellar opening;

(c) Locating the keratoprosthesis as described herein within the lamellar pocket so that the optic overlies the posterior opening, fixing the pocket closed and optionally creating a conjunctival flap; and

(d) Trephination of the tissues that lie anterior to the keratoprosthesis optic so as to expose the keratoprosthesis optic as a fall thickness corneal replacement.

Preferably, the posterior lamellar opening is formed by central trephination through the posterior lamella into the anterior chamber.

These and other features of the invention will become apparent from the following detailed description of the invention.

### DETAILED DESCRIPTION OF THE INVENTION

General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively, and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

As used herein the term “derived” and “derived from” shall be taken to indicate that a specific integer may be obtained from a particular source albeit not necessarily directly from that source.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Description

The present invention is based on a finding that insertion of a hydrogel keratoprosthesis into a patient's eye leads to an in situ deformation of the keratoprosthesis optic when exposed as the full-thickness replacement of the cornea. The power of a human biological cornea in its position within the eye is approximately 42 Diopters (“D”). A hydrogel artificial cornea, such as previously described in U.S. Pat. Nos. 5,300,116 and 5,458,819, (unlike earlier, rigid (and thus complication-prone) keratoprostheses), designed to deliver this power in situ with no allowance having been made for in situ alterations, would demonstrate an increased power in situ, of approximately 58 D, as a result of the shortening of optic radii of curvature that occurs in situ and which would cause a considerable overcorrection for most phakic or pseudophakic patients. The applicant has confirmed that this effect is related to both the material properties of the artificial cornea and to ocular factors in such a way that the degree of the in situ power change can be predicted and manipulated so that an in situ power desirable for the individual patient can be obtained.

According to one form of the invention, there is provided an improved keratoprosthesis comprising a keratoprosthesis prepared with such properties and radii that when implanted into a patient it assumes the optic radii of curvature for a desirable refractive outcome.

The present invention provides significant advantages over prior art keratoprostheses in that it can be given a defined, accurate ‘test power’ that is known to produce a desired ‘in situ predicted power’ selected for an individual and that can be further customised after implantation. Thus, the improved keratoprosthesis significantly improves the likelihood of a good unaided visual acuity when located in situ and minimises the degree of subsequent postoperative corrections to achieve best-corrected visual acuity. Further, unaided visual acuities provided by such an artificial cornea are believed to be better and more rapidly stable than the unaided visual acuity outcomes presented in the literature for standard donor corneal grafts.

According to the invention, the specifications of the improved keratoprosthesis are set so as to lead to a refractive power in the order of 42 D in situ in phakic or pseudophakic patients and appropriately higher in aphakic recipients. The keratoprostheses are manufactured with such radii of curvature that, when implanted in situ in the eye, the post-implantation changes that occur in the radii of curvature render them ideal for the optical outcome desired, i.e. such an improved keratoprosthesis located in situ has a refractive power in situ that is dependent both upon the radii of curvature as originally lathed (that would produce an in-eye power of the desired range if no further alteration occurred), and upon the post-implantation changes that have been determined to occur and which occur to a largely predictable degree.

In a highly preferred form of the invention the improved keratoprosthesis is prepared in such a manner that the theoretical refractive power of the keratoprosthesis at an air-aqueous interface is preferably greater than about 20 D but less than 48 D when introduced into the eye. Further, the invention encompasses the recognition that a post-implantation deformation occurs and that the original lathed radii require to be set such that the post-implantation deformation that occurs in situ causes the radii to alter so that the final optical power, preferably in the range 42-62 D (for phakic/pseudophakic and aphakic patients) is achieved. Most preferably, the refractive power of the keratoprosthesis when tested in an air-air or aqueous-aqueous system, is close to plano, but with such radii of curvature that, after implantation in situ, the full desired power, generally 40-46 D for phakic/pseudophakic patients and 56-62 for aphakic patients, is achieved. It will be clear to those skilled in the art of standard lens formulae that there are several combinations of anterior and posterior radii of curvature that would, by lens equivalence, achieve the same test power in a given medium or pair of media. The anterior and posterior radii of curvature of the keratoprosthesis optic surfaces both have a preferable range of 6.0 mm to infinity. Desirably, the improved keratoprosthesis results in an unaided visual acuity of 20/200 (6/60) or better, with 20/20 (6/6) being highly preferred, however being understood to depend in part upon other individual factors and limited in some cases by unrelated pathologies.

In an embodiment of the invention the keratoprosthesis is prepared in the same manner as described in Australian patent 650156, U.S. Pat. Nos. 5,300,116 or 5,458,819, herein incorporated by reference. Briefly stated, the keratoprosthesis of the present invention comprises first a peripheral circular portion as the rim of the device, made by homopolymerisation or copolymerisation in a solution of HEMA using large excess of water and resulting in a non-transparent hydrogel sponge. In the central aperture of the said sponge, a transparent circular portion is created by the subsequent polymerisation or copolymerisation in solution of HEMA using lower amounts of water than in the case of the sponge formation. Alternatively, the central portion can be firstly created, and followed by the production of the spongy rim in the manner disclosed above. Regardless of the succession of the two processes, through this sequential two-stage polymerisation performed in the same mould, a tight and intimate attachment between the materials in the two portions of keratoprosthesis is achieved due to the fact that, during the second stage, the monomer mixture firstly penetrates to some extent into the pre-existent polymer matrix and only afterwards undergoes polymerisation. As a result, an interpenetrating polymer network (IPN) region is formed along the boundary between the central portion and rim.

The improved keratoprosthesis may be conveniently formed from PHEMA, or from mixtures of HEMA and other hydrophilic and/or hydrophobic monomers as disclosed in the patents of the prior art. PHEMA hydrogel sponges allow the invasion of cells from the host corneal stroma into the pores of the sponge. Representative of other monomers useful in conjunction with HEMA as comonomers of the present invention are:

a. Other hydroxylated methacrylates and acrylates, such as 2-hydroxyethyl acrylate, 2-hydroxypropyl methacrylate and acrylate, glycerol methacrylates and acrylates, and many others well known in the art, as such or mixtures thereof.

b. Acrylamide derivatives and N-vinylpyrrolidone, and various combinations thereof.

c. Hydrophobic methacrylates and acrylates, such as methyl methacrylate or other members of the aliphatic homologue series, and 2-alkoxyethyl methacrylates and acrylates.

d. Other hydrophobic monomers, such as vinyl acetate and vinyl propionate.

Whilst the hydrophilic monomers may be added to HEMA in any proportion from 0 to 50% by weight based on the total amount of monomers, the hydrophobic monomers may be added in a proportion not higher than 5%, in order to avoid the occurrence of phase separation prior to polymerisation.

Where the keratoprosthesis is prepared in such a form and from such material (as described above) the radii of curvature of the optic of the improved keratoprosthesis are selected such that the alterations predicted to occur once the optic is exposed to the conditions of the implantation site as a full-thickness corneal replacement, will result in the radii assuming the appropriate lengths such that the required optical power is produced, ie approximately 42 D for phakic or pseudophakic patients or 56 D for aphakes. It will be understood, however, that any other desired in situ power is achievable through the appropriate determination of the initial radii during manufacture, these being determined through knowledge of standard lens formulae, material characteristics of the keratoprosthesis material in comparison with native cornea, and correction factors determined by the applicant. In all cases, it is the desired final outcome that the sum total of the power in the optical system as a whole, will be as near to that required for emmetropia as possible, with the proviso that intentional refractive errors, such as a low myopia outcome, could be selected if required, for example if a patient requested monovision, with the eye receiving the keratoprosthesis to be corrected for near vision.

The power of a keratoprosthesis before implantation (the ‘test power’ in a given medium or pair of media) is dependent on the refractive index of the polymer, of the medium or media and the anterior and posterior radii of curvature of the keratoprosthesis. The power in situ includes those factors mentioned above as well as the dimension and shape of the anterior and posterior lamellar openings in the adjacent corneal tissue, the intraocular pressure of the eye, the hydration and temperature of the exposed optic and mechanical constraints of the material including Poisson's ratio and Young's modulus. Poisson's ratio is the ratio of the lateral expansion per unit breadth to the longitudinal change per unit length. Young's modulus refers to the applied force per cross-section area for the longitudinal change per unit length (strain).

The refractive power of the lens in the specified test media may be calculated theoretically and measured using standard techniques and instruments such as lens analysers and lensometers well known to the prior art. The test power that relates to the desired in situ power is known through a correction factor developed from theoretical principles, tested through a computer model and confirmed empirically in human patients.

The desirable thickness of the keratoprosthesis is approximately the same as the thickness of the host cornea tissue into which it is to be implanted, the preferable range being 0.35-0.7 mm. The thickness is such that the keratoprosthesis should withstand the forces exerted during implantation with no tearing or permanent deformation. Preferably the thickness of the keratoprosthesis is greater than 0.40 mm thick. In a highly preferred form the keratoprosthesis is between 0.50 and 0.65 mm thick with a keratoprosthesis of a thickness of about 0.60 mm being desirable.

Variation of the anterior and posterior radii of curvature of the keratoprosthesis may also impact on the power of the improved keratoprosthesis. Preferably the radii of curvature of the keratoprosthesis optic are selected in association with the other factors impacting on the power of the keratoprosthesis to produce a keratoprosthesis with the desired refractive power which when inserted in the patient's eye leads to an appropriate refractive power for the individual's requirements, most commonly close to 42 D in phakic patients. Allowing for standard physiology and morphology of the eye, the anterior and posterior radii of curvature are preferably greater than 6.0 mm in order to accommodate individual requirements. Preferably, the anterior and posterior optic radii are set during manufacture to between 6.5 mm and 10 mm. More preferably, the radii are between 8.0 mm and 9.0 mm. It will be clearly understood that this does not require keratoprostheses to be individually made to order, but allows for a range of devices to be available for supply such that the device whose test power is predicted to give the desired refractive outcome for the individual, to be selected.

While the anterior and posterior radii of curvature may be of different lengths, for example, the anterior radius might be 9.0 mm and the posterior radius might be 8.5 mm, preferably the anterior and posterior radii are the same length such 9.0 mm, such an optical lens being designated ‘plano’ upon testing in a uniform medium, but delivering the desired optical power once in situ in the air-aqueous interface of the cornea.

The desirable diameter of the keratoprosthesis is preferably in the range 6.0 mm to 9.0 mm. Preferably the diameter is 7.0 mm.

It should be appreciated that by changing the anterior and posterior radii of curvature, the thickness of the keratoprosthesis and the diameter of the keratoprosthesis optic that is fully exposed to the intraocular pressure, it is possible to alter the power of the keratoprosthesis in such a manner to account for different needs of aphakic, pseudophakic and phakic patients. For example, in one form of the invention, there is provided an improved keratoprosthesis for implantation into an aphakic patient, said keratoprosthesis including, as an example, but not to be taken exclusively, an anterior radius of curvature of 8.0 mm and a posterior radius of curvature of 8.0 mm, with a thickness of about 0.60 mm and a diameter of between 6.0 mm and 8.0 mm. In one alternate form of the invention there is provided an improved keratoprosthesis for implantation into phakic or pseudophakic patients, said keratoprosthesis including an anterior radius of curvature of 9.0 mm and a posterior radius of curvature of 9.0 mm, with a thickness of about 0.60 mm and a the diameter of between 6.0 mm and 8.0 mm. Furthermore, different radii can be chosen to suit eyes of differing dimension, and the desired outcomes for individual patients such as a deliberate over-correction resulting in myopia if a patient desires a given eye to be corrected primarily for near vision.

After an improved keratoprosthesis is inserted into a patient's eye it may in some instances be necessary to make minor adjustments to the morphology of the keratoprosthesis to enhance visual acuity. When introduced into a patient's eye the keratoprosthesis may be tailored to the precise needs of the patient using any treatment known in the art.

According to a second form of the invention, there is provided a method of implanting a keratoprosthesis into a patient, said method comprising the steps of:

(a) Creating a lamellar corneal pocket and by central trephination through the posterior lamella into the anterior chamber, providing a posterior lamellar opening;

(b) Positioning the keratoprosthesis as described herein within the lamellar pocket so that the optic overlies the posterior opening, suturing the pocket closed and optionally creating a conjunctival flap; and

(c) Trephination of the tissues that lie anterior to the keratoprosthesis optic so as to expose the keratoprosthesis optic as a full thickness corneal replacement.

Preferably there is a 6 to 16 week time delay between the performance of steps (a) and (b) in the method and step (c).

During the process of insertion of the keratoprosthesis trephination of the lamella is achieved using standard procedures. Preferably, trephination of the anterior and posterior lamella lead to openings between 2.0 mm and 4.0 mm in size. Most preferably the openings are between 2.0 mm and 3.0 mm in size with approximately 3.0 mm being highly desirable.

It is desirable that any opening so formed is circular in shape, since any other shaped opening may allow alterations of optic radii of curvature to differ in different axes, so causing astigmatism. Appropriate surgical and post-operative management should aim to maintain these circular openings such that their shape and dimension remains constant. This may require specific therapeutic regimens in patients at high risk of corneal melting.

In a desired form of the invention the keratoprosthesis that is introduced into the patient's eye is of a slightly higher corrective power than required to produce a power of 42 D when located in situ as most patients prefer myopia to hyperopia, it being better to risk a degree of myopia to a hyperopic outcome; further, myopia can be simpler to correct by subsequent standard techniques. Preferably, the refractive power of the improved keratoprosthesis in situ is less than 10 D greater than the normal refractive power of the cornea, except where a higher power has been preferentially selected, such as for an aphakic patient. Even more preferably, the in situ over-correction of the keratoprosthesis is less than 5 D with between 1 D and 4 D being strongly desired.

According to preferred embodiment of the method of the invention, there is provided a method of implanting a keratoprosthesis into a patient, said method including the steps of:

(a) Creating a lamellar corneal pocket and trephining centrally through the posterior lamella into the anterior chamber, providing a posterior lamellar opening of approximately 3.0 mm;

(b) Positioning the keratoprosthesis as herein described within the lamella pocket, suturing the pocket closed and creating a conjunctival flap; and, during the deferred second stage of surgery,

(c) Trephination of tissues anterior to the keratoprosthesis optic to create an opening of approximately 3.0 mm to expose the keratoprosthesis as a full thickness corneal replacement.

In a highly preferred form of the invention the keratoprosthesis used in this method is selected according to whether the patient is aphakic, phakic or pseudophakic and including consideration of other ocular factors such as axial length. Where the patient is aphakic the keratoprosthesis generally chosen has an anterior radius of curvature of 8.0 mm and a posterior radius of curvature of 8.0 mm, with a thickness of about 0.60 mm and a diameter of between 6.0 mm and 8.0 mm. Where the keratoprosthesis is for implantation into a phakic patient or a pseudophakic patient, the keratoprosthesis has an anterior radius of curvature of 9.0 mm and a posterior radius of curvature of 9.0 mm, with a thickness of about 0.60 mm and a the diameter of between 6.0 mm and 8.0 mm. However, the above should be taken only as examples and keratoprostheses with different specifications may equally be selected where indicated.

In a further embodiment of the invention, the method comprises the steps of preparing the keratoprosthesis in the known manner, lathing the keratoprosthesis to the desired dimensions depending on the power requirements of the patient, (allowing for the increase in power of the keratoprosthesis on implantation) and implantation of the keratoprosthesis, (considering the required dimensions of the anterior and posterior corneal lamellar openings to produce the correct power requirements for the patient) followed by correction of the implanted keratoprosthesis if necessary.

### Best Mode for Carrying out the Invention

The following Examples serve to more fully describe the manner of using the above-described invention, as well as set forth the best modes contemplated for carrying out various aspects of the invention. It is understood that these Examples in no way serve to limit the true scope of this invention, but rather are presented for illustrative purposes. The references cited herein are expressly incorporated by reference.

### EXAMPLE 1

To test the power of prior art keratoprosthesis in situ keratoprostheses produced according to the methods described in Australian patent 650156, U.S. Pat. Nos. 5,300,116 or 5,458,819 were manufactured, inserted into patients' eyes and various measurements were taken. The results of the measurements are summarised below:

Patient 1: This aphakic patient received a keratoprosthesis 0.45 mm thick, anterior radius of curvature r=8.0 mm, posterior radius of curvature r=8.5 mm. This was calculated theoretically to give 42 D in situ had there been no expected alteration in radii in situ, but predicted to give a greater power than this once in place. The posterior lamella opening was approximately 2.0 mm, while the anterior lamella opening was approximately 3.0 mm.

The actual power in situ was estimated to be approximately 58 D (ie approximately that required for an aphakic patient, but considerably greater than that a phakic patient would have required). Consistent with this was the finding that the patient's unaided visual acuity was approximately 20/120-20/80 (6/36-6/24), which, allowing for pre-existing glaucomatous damage, indicated that the patient's refractive status was close to the ideal.

Patient 2: This aphakic patient received a keratoprosthesis 0.49 mm thick, anterior radius of curvature r=8.0 mm, posterior radius of curvature r=8.5 mm, again, that would have provided 42 D in situ if no in situ alteration occurred, but with in situ changes being expected to increase the effective in situ power to that required to compensate for the lack of a crystalline lens.

This gave an outcome approximately similar to that of Patient 1, although the anterior lamella opening was approximately 2.5 mm and was vertically oval, and this resulted in irregular, with-the-rule myopic astigmatism that was confirmed by post-operative keratoscopy readings.

Patient 3: This phakic patient received a keratoprosthesis 0.50 mm thick, of anterior radius of curvature r=8.0 mm, posterior radius of curvature r=8.5 mm. In this case, although the radii of the keratoprosthesis as manufactured would give an in situ power predicted from lens formulae without account of in situ changes, of 42 D, the effect of in situ radii shortening (optic steepening) caused an increase in power as for patients 1 & 2, but in this case, undesirable, as a natural crystalline lens was still present. The additional power induced a highly myopic outcome in this patient (20/200, or 6/60 unaided, N10 at 10 cm), consistent with an in situ power being considerably above 42 D.

Patient 4: This pseudophakic patient received a keratoprosthesis identical to that given to patient 3, 0.52 mm thick, anterior radius of curvature r=8.0 mm, posterior radius of curvature r=8.5 mm. Both posterior and anterior lamella openings were approximately 3.5 mm. The patient's refractive outcome was highly myopic, of about −18 D, with imaging confirming an increase in optic radius of curvature, a deepening of the anterior chamber and high simulated keratoscopy readings. In this patient, a rigid contact lens allowed a negative tear lens to compensate for the myopia, and resulted in the final desired outcome of 20/20 −2 (6/6-2) and a high degree of patient satisfaction.

These results were consistent with the view that a “42 D” keratoprosthesis was really about 57-58 D in situ and corroborated predictions from computer simulations. Accordingly, subsequent phakic or pseudophakic patients received keratoprosthesis in which the optical parameters had been manufactured to allow for post-implantation changes, to minimise the extent of post-operative refractive correction required, while aphakic patients continued to receive devices intended to deliver the additional optical power that their aphakic state required.

Patient 5: This pseudophakic patient received a keratoprosthesis 0.55 mm thick, of anterior radius of curvature r=9.0 mm, posterior radius of curvature r=9.0 mm. Although visual acuity was limited postoperatively by other pathologies, the keratoprosthesis appeared satisfactory.

Follow-up of patients with artificial corneas of the preferred optic radii of curvature is currently too short for final refractive status to have been determined but interim findings suggest that the devices produce outcomes in the range of low myopia to low hyperopia as desired and suitable for further refractive correction during the postoperative period using any names described in the prior art.

### EXAMPLE 2

The improved keratoprosthesis was prepared as described in the prior art and was lathed to the desired dimensions. For a keratoprosthesis to be used in an aphakic patient, the keratoprosthesis comprised an anterior radius of 8.0 mm, a posterior radius of 8.0 mm, a thickness of 0.60 mm and a diameter of 7.0 mm. The keratoprosthesis was inserted into the patient by cutting a lamellar corneal pocket and trephining centrally through the posterior lamellar into the anterior chamber, providing a posterior lamellar opening of 3.0 mm. The keratoprosthesis was positioned within the pocket, which was then sutured closed. The anterior tissues were subsequently trephined to expose the keratoprosthesis as a fall thickness corneal replacement. The anterior lamellar opening was 3.0 mm. The dimensions of the keratoprosthesis and the lamellar opening dimensions provide for an actual power in situ of approximately 60 D, suited for the aphakic patient.

Similarly, but alternatively, if the keratoprosthesis was manufactured and inserted as above, but with anterior and posterior radii of curvature lathed to 9.0 mm and 9.0 mm, the effective in situ power would be about 15 D less than in the prior example, and thus appropriate for phakic or pseudophakic patients.

While an advantageous and preferred embodiment of the present invention has been selected and described as an illustration of the invention, it should be understood by those skilled in the art that changes and adaptations can be made therein without departing from the scope of the invention as defined in the appended claims.

1. An improved keratoprosthesis comprising a keratoprosthesis prepared with such properties and radii that when implanted into a patient it assumes the optic radii of curvature for a desirable refractive outcome.

2. An improved keratoprosthesis according to claim 1 wherein the specifications of the improved keratoprosthesis are selected to lead to a refractive power in the order of 42 D in situ in phakic or pseudophakic patients and appropriately higher in aphakic recipients.

3. An improved keratoprosthesis according to claim 1 wherein the keratoprostheses are manufactured with such radii of curvature that, when implanted in situ in the eye, the post-implantation changes that occur in the radii of curvature, render them suitable for the optical outcome desired.

4. An improved keratoprosthesis according to claim 1 wherein the improved keratoprosthesis is prepared in such a manner that the refractive power of the keratoprosthesis at an air-aqueous interface is preferably greater than about 20 D but less than 48 D.

5. An improved keratoprosthesis according to claim 1 wherein the refractive power of the keratoprosthesis when tested in an air-air or aqueous-aqueous system, is close to plano, but with such radii of curvature that, after implantation in situ, the full desired power, generally 40-46 D for phakic/pseudophakic patients and 56-62 D for aphakic patients, is achieved.

6. An improved keratoprosthesis according to claim 1 wherein the anterior and posterior radii of curvature of the keratoprosthesis optic surfaces are greater than or equal to 6.0 mm.

7. An improved keratoprosthesis according to claim 1 wherein improved keratoprosthesis is formed from poly(2-hydroxyethyl methacrylate), commonly designated as PHEMA.

8. An improved keratoprosthesis according to claim 1 wherein improved keratoprosthesis is formed from mixtures of 2-hydroxyethyl methacrylate, commonly designated as HEMA and other hydrophilic and/or hydrophobic monomers.

9. An improved keratoprosthesis according to claim 1 wherein the keratoprosthesis is prepared in such a form and from such material that the radii of curvature of the optic of the improved keratoprosthesis are selected such that the alterations predicted to occur once the optic is exposed to the conditions of the implantation site as a full-thickness corneal replacement, will result in the radii assuming the appropriate lengths such that the required optical power is produced, which is approximately 42 D for phakic or pseudophakic patients or 56 D for aphakes.

10. An improved keratoprosthesis according to claim 1 wherein the desirable thickness of the keratoprosthesis is approximately the same as the thickness of the host cornea tissue into which it is to be implanted

11. An improved keratoprosthesis according to claim 10 wherein the thickness ranges from 0.35 to 0.7 mm.

12. An improved keratoprosthesis according to claim 10 wherein the thickness of the keratoprosthesis is greater than 0.40 mm thick.

13. An improved keratoprosthesis according to claim 10 wherein the keratoprosthesis is between 0.50 and 0.65 mm thick

14. An improved keratoprosthesis according to claim 10 wherein the keratoprosthesis is 0.60 mm thick.

15. An improved keratoprosthesis according to claim 1 wherein the radii of curvature of the keratoprosthesis optic are selected in association with the other factors impacting on the power of the keratoprosthesis to produce a keratoprosthesis with the desired refractive power which when inserted in the patient's eye leads to an appropriate refractive power for the individual's requirements, most commonly close to 42 D in phakic patients.

16. An improved keratoprosthesis according to claim 15 wherein the anterior and posterior radii of curvature are preferably greater than 6.0 mm in order to accommodate individual requirements.

17. An improved keratoprosthesis according to claim 15 wherein the anterior and posterior optic radii are set during manufacture to between 6.5 mm and 10 mm.

18. An improved keratoprosthesis according to claim 15 wherein the radii are between 8.0 mm and 9.0 mm.

19. An improved keratoprosthesis according to claim 15 wherein the anterior and posterior radii are of the same length.

20. An improved keratoprosthesis according to claim 1 wherein the keratoprosthesis has a diameter of between 6.0 mm and 9.0 mm.

21. An improved keratoprosthesis according to claim 20 wherein the Keratoprosthesis has a diameter of 7.0 mm.

22. An improved keratoprosthesis according to claim 1 wherein said keratoprosthesis has an anterior radius of curvature of 8.0 mm and a posterior radius of curvature of 8.0 mm, with a thickness of about 0.60 mm and a diameter of between 6.0 mm and 8.0 mm.

23. An improved keratoprosthesis according to claim 1 wherein said keratoprosthesis has an anterior radius of curvature of 9.0 mm and a posterior radius of curvature of 9.0 mm, with a thickness of about 0.60 mm and a diameter of between 6.0 mm and 8.0 mm.

24. A method of implanting a keratoprosthesis into a patient, said method comprising the steps of:
a. Creating a lamellar corneal pocket and, by central trephination through the posterior lamella into the anterior chamber, providing a posterior lamellar opening;
b. Positioning a keratoprosthesis according to claim 1 within the lamellar pocket so that the optic overlies the posterior opening, suturing the pocket closed and optionally creating a conjunctival flap; and
c. Trephination of the tissues that lie anterior to the keratoprosthesis optic so as to expose the keratoprosthesis optic as a full thickness corneal replacement.

25. A method according to claim 24 wherein there is a 6 to 16 week time delay between the performance of steps (a) and (b) and step (c).

26. A method according to claim 24 wherein trephination of the anterior and posterior lamella lead to openings between 2.0 mm and 4.0 mm in size.

27. A method according to claim 26 wherein the openings are between 2.0 mm and 3.0 mm in size

28. A method according to claim 26 wherein the openings are approximately 3.0 mm.

29. A method according to claim 24 wherein the keratoprosthesis that is introduced into the patient's eye is of a slightly higher corrective power than required to produce a power of 42 D when located in situ.

30. A method according to claim 29 wherein the refractive power of the improved keratoprosthesis in situ is less than 10 D greater than the normal refractive power of the cornea.

31. A method according to claim 29 wherein the in situ over-correction of the keratoprosthesis is less than 5 D

32. A method according to claim 29 wherein the in situ over-correction of the keratoprosthesis is between 1 D and 4 D.

33. A method of implanting a keratoprosthesis into a patient, said method including the steps of:
a. Creating a lamellar corneal pocket and trephining centrally through the posterior lamella into the anterior chamber, providing a posterior lamellar opening of approximately 3.0 mm;
b. Positioning a keratoprosthesis according to claim 1 within the lamella pocket, suturing the pocket closed and creating a conjunctival flap; and, during the deferred second stage of surgery,
c. Trephination of tissues anterior to the keratoprosthesis optic to create an opening of approximately 3.0 mm to expose the keratoprosthesis as a full thickness corneal replacement.

34. A method according to claim 33 wherein the keratoprosthesis has an anterior radius of curvature of 8.0 mm and a posterior radius of curvature of 8.0 mm, with a thickness of about 0.60 mm and a diameter of between 6.0 mm and 8.0 mm.

35. A method according to claim 33 wherein the keratoprosthesis has an anterior radius of curvature of 9.0 mm and a posterior radius of curvature of 9.0 mm, with a thickness of about 0.60 mm and a diameter of between 6.0 mm and 8.0 mm.

36. The improved keratoprosthesis of claim 1 that allows assumption of optic radii of curvature in situ for desirable refractive outcome that can be adjusted after implantation.

37. The improved keratoprosthesis of claim 36, wherin said adjustment is by laser ablation.

38. The method of implanting a keratoprosthesis of claim 24, wherein said keratoprosthesis allows assumption of optic radii of curvature in situ for desirable refractive outcome that can be adjusted.

39. The method of claim 38, wherein said adjustment is by laser ablation.
